(19) United States
(12) Patent Application Publication
(10) Pub. No.: US 2025/0228998 A1
(43) Pub. Date: Jul. 17, 2025

(54) Anionic Citrate-Based Biomaterials

(71) Applicant: Acuitive Technologies, Inc., Allendale, NJ (US)

(72) Inventors: Minh-Tuan Richard Tran, Ho-Ho-Kus, NJ (US); Ruqiang Lu, Ramsey, NJ (US)

(73) Assignee: Acuitive Technologies, Inc., Allendale, NJ (US)

(21) Appl. No.: 18/736,005

(22) Filed: Jun. 6, 2024

Related U.S. Application Data
(60) Provisional application No. 63/471,567, filed on Jun. 7, 2023.

(51) Int. Cl. A61L 27/18; A61L 27/46; A61L 27/54; A61L 27/56; A61L 27/58

(52) U.S. Cl. CPC A61L 27/18; A61L 27/46; A61L 27/54; A61L 27/56; A61L 27/58; A61L 2300/252; A61L 2300/414; A61L 2400/18; A61L 2430/02

(57) ABSTRACT

The present disclosure provides anionic citrate-based composition for use in regenerative applications. The disclosed composition may take the form of a scaffold and generally includes (i) a citrate component, (ii) a diol, and (iii) an anionic moiety. The anionic moiety may include one or more of a sulfate, sulfonate, phosphate, phosphonate, sulfamate, or carboxylic acid, The sulfate and/or sulfonate containing moiety may be conjugated via an amidation reaction, esterification, and/or substitution reaction with a sulfur trioxide complex. A peptide may be conjugated to a surface of the composition.

### CROSS-REFERENCE TO RELATED APPLICATION

The present application claims priority benefit to a U.S. provisional patent application entitled “Anionic Citrate-Based Biomaterials,” filed on Jun. 7, 2023, and assigned Ser. No. 63/471,567. The entire content of the foregoing U.S. provisional patent application is incorporated herein by reference.

### BACKGROUND

### 1. Technical Field

The present disclosure is directed to anionic citrate-based compositions for controlled binding and release of proteins for regenerative engineering applications.

### 2. Background Art

Despite bone's capacity to regenerate after injury, delayed healing or nonunion is a significant clinical challenge in orthopedics (Chen C. H., Hsu E. L., Stupp S. I. Supramolecular self-assembling peptides to deliver bone morphogenetic proteins for skeletal regeneration. Bone. Volume 141, December 2020, 115565). Out of the 8 million fracture repair cases which occur annually in the U.S., over 10% resulted in nonunion and significantly higher rates have been reported for smokers, diabetics, and osteoporotic patients (Buza J. A. 3rd; Einhorn T. Bone healing in 2016. Clinical Cases Mineral and Bone Metabolism 2016, 13(2), 101-105).

Autogenous bone grafting from the iliac bone has been considered the clinical gold standard treatment for bone formation in the repair of large bone defects and fusion applications (Dohzono S., Imai Y., Nakamura H., Wakitani S., Takaoka K. Successful spinal fusion by E. coli-derived BMP-2-adsorbed porous beta-TCP granules: a pilot study. Clinical Orthopaedics and Related Research. December 2009, 467(12), 3206-3212). However, donor site deformity, pain, infection, and supply limitations are major disadvantages associated with the harvesting of iliac bone graft (Arrington E. D., Smith W. J., Chambers H. G., Bucknell A. L., Davino N. A. Complications of iliac crest bone graft harvesting. Clinical Orthopaedics and Related Research. 1996, 329, 300-309). These limitations in combination with developments in bone biology have driven researchers towards alternative approaches using bioactive molecules and materials.

For example, bone morphogenic protein (BMP) is a naturally occurring bioactive molecule capable of ectopic bone formation (Urist M. R. Bone-formation by Autoinduction. Science. 1965, 150(3698), 893-899). While healing rates as high as 92% have been reported in nonunion patients (Zhou Y. Q., Tu H. L., Duan Y. J., Chen X. Comparison of bone morphogenetic protein and autologous grafting in the treatment of limb long bone nonunion: a systematic review and meta-analysis. Journey of Orthopedic Surgery and Research. 2020, 15(1), 288), adverse effects such as inflammatory reactions, soft tissue hematomas, bone cysts, ectopic bone formation, cancer, retrograde ejaculation, infections, and radiculopathy have been connected to BMP treatment (El Bialy I., Jiskoot W., Reza Nejadnik M. Formulation, Delivery and Stability of Bone Morphogenetic Proteins for Effective Bone Regeneration. Pharmaceutical Research. 2017, 34(6), 1152-1170). It has been widely reported that the main reason behind these adverse effects is the high dose of administered BMP (Mroz T. E., Wang J. C., Hashimoto R., Norvell D. C. Complications related to Osteobiologics use in spine surgery a systematic review. Spine. 2010, 35(9), S86-S104.). For example, Medtronic's INFUSE bone graft kit utilizes 1.5 mg/mL of BMP-2, which exceeds one million times the physiological concentrations during normal bone repair conditions (Gamradt S. C., Lieberman J. R. Genetic modification of stem cells to enhance bone repair. Annals Biomedical Engineering. 2004, 32(1), 136-147).

It has been shown that the efficacy and some of the reported side effects of BMPs can be controlled by improving their delivery system. Current BMP delivery systems are based on collagen due to its biocompatibility and ability to be fabricated as powders, films, gels, fibers, and absorbable sponges (Lee S. H., Shin H. Matrices and scaffolds for delivery of bioactive molecules in bone and cartilage tissue engineering. Advanced Drug Delivery Review. 2007, 59(4-5), 339-359.). However, it has been shown in vivo that only 5% of BMP remains within collagen sponges after 2 weeks due to an initial burst release (Geiger M., Li R. H., Friess W. Collagen sponges for bone regeneration with rhBMP-2. Advanced Drug Delivery Review. 2003, 55(12), 1613-1629).

To address this limitation in collagen-based BMP delivery, significant research activity has been focused on the design of biomaterial delivery systems with improved protein binding capabilities to reduce the requisite dosage of BMP-2. Many studies have attempted to reduce the dosage and decrease the release rate of BMP-2 by binding heparin to their carriers. Heparin is a highly sulfated glycosaminoglycan that is negatively charged, which causes electrostatic interactions with growth factors like BMP-2, forming a polyelectrolyte complex (Terauchi M., Tamura A., Tonegawa A., Yamaguchi S., Yoda T., Yui N. Polyelectrolyte complexes between polycarboxylates and BMP-2 for enhancing osteogenic differentiation: effect of chemical structure of polycarboxylates. Polymers. 2019, 11(8), 1327-1327). However, the main disadvantage to using heparin to attract growth factors is its anticoagulant effect, which could increase the risk of complications during surgical operation and healing (Terauchi et al.).

For at least the foregoing reasons, a need exists for biocompatible compositions with improved protein binding and controlled protein release capabilities for regenerative engineering applications, e.g., replacements for injured tissues.

### SUMMARY

According to the present disclosure, highly advantageous anionic citrate-based compositions are fabricated and utilized for controlled binding and release of proteins through polyelectrolyte complexes.

In exemplary embodiments, the disclosed composition includes (i) a citrate component, (ii) a polyol, and (iii) an anionic moiety. The citrate component may comprise one or more of citric acid, citrate, or ester of citric acid. The polyol may comprise a diol, e.g., one or more of butanediol, hexanediol, octanediol, or polyethylene glycol. Other exemplary polyols contemplated according to the present disclosure include one or more of glycerol, beta-glycerol phosphate, or xylitol.

The anionic moiety may include one or more of sulfate, sulfonate, phosphate, phosphonate, sulfamate, or carboxylic acid. In certain exemplary embodiments, the sulfate and/or sulfonate containing moiety may further include one or more of hydroxyl, amine, or carboxylic acid. In other exemplary embodiments, the sulfate and/or sulfonate containing moiety may include one or more of taurine, isethionic acid, or sodium 2-carboxyethane-1-sulfonate.

In some exemplary embodiments, the sulfate and/or sulfonate containing moiety may be conjugated via amidation reaction, esterification, and/or substitution reaction with a sulfur trioxide complex. In a more specific example, the amidation reaction may be assisted by 1-ethyl-3-(-3-dimethylaminopropyl) carbodiimide hydrocholoride (EDC).

In some embodiments, the phosphate and phosphonate containing moiety may include one or more of hydroxyl, amine, or carboxylic acid. In a more specific example, the phosphate and phosphonate containing moiety may include 3-(phosphonooxy) propanoic acid or 2-hydroxyethyl phosphate. In other exemplary embodiments, the anionic moiety may further include one or more of alkene, alkyne, halogen, borate, or azide.

In exemplary embodiments, the composition may further include a peptide conjugated to a surface of the composition. In some embodiments, the peptide is a protein binding peptide. In other embodiments, the peptide is a protein mimicking peptide.

In exemplary embodiments, the composition may further include a particulate inorganic material. The particulate inorganic material may include one or more of hydroxyapatite, tricalcium phosphate, biphasic calcium phosphate, or Bioglass. In some embodiments, the particulate inorganic material is present in an amount between 10 and 60 wt.-% of the composition. In certain embodiments, the particulate inorganic material is micro or nano-sized. In some exemplary embodiments, the particulate inorganic material is rod-shaped. In other exemplary embodiments, the particulate inorganic material is fiber-shaped.

In some exemplary embodiments, a scaffold can be formed at least in part from the disclosed composition, wherein the scaffold is biodegradable. In some embodiments, the scaffold is 50-90% porous. In exemplary embodiments, the scaffold contains a gradient porous structure. In some exemplary embodiments, the composition binds proteins through electrostatic interactions. In some embodiments, the protein is one or more of bone morphogenic protein (BMP), basic fibroblast growth factor (bFGF), transforming growth factor beta (TGF-β1), vascular endothelial growth factor (VEGF), platelet derived growth factor (PDGF), and/or epidermal growth factor (EGF). In a more specific example, the BMP may include BMP-2, BMP-4, or BMP-7.

Additional features, functions, and benefits of the disclosed citrate-based compositions will be apparent from the following description of exemplary embodiments.

### BRIEF DESCRIPTION OF THE FIGURES

To assist those of skill in the art in making and using the disclosed citrate-based compositions, reference is made to the accompanying figures, wherein:

FIG. 1 is a schematic depiction of the chemical structure of POXC-IA, as described herein;

FIG. 2 is a schematic depiction of the chemical structure of POXC-IA with an associated ¹H NMR spectra that demonstrates the successful addition of IA, as described herein;

FIG. 3 is a schematic depiction of conjugation of a sulfates/sulfonates to the surface of a citrate-based biomaterial using free carboxylic acid (conjugation “A”) or an hydroxyl group (conjugation “B”);

FIG. 4A-FIG. 4D comprise four panels, as follows:

FIG. 4A shows poly (octamethylene-xylitol citrate) (POXC) scaffolds that are surface modified to present sulfate and/or sulfonate groups;

FIG. 4B shows high-resolution X-ray photoelectron spectroscopy (XPS) spectra of S2p;

FIG. 4C shows sulfate/sulfonate content obtained from the integration of high-resolution XPS spectra of S2p and C1s (which is defined as the ratio of S atom and C atom contents; the C atom content for POXC-IA-Ta was corrected via the subtraction of the C atom from taurine); and

FIG. 4D shows surface zeta-potentials of INFUSE collagen (Medtronic plc, Minneapolis, MN), POXC, POXC-IA-Ta and POXC-IA-SO3Na scaffolds under different pHs.

FIG. 5 is a plot showing cumulative rhBMP-2 release from INFUSE collagen scaffolds and sulfate/sulfonate modified POXC scaffolds;

### DESCRIPTION OF EXEMPLARY EMBODIMENTS

As noted above, the present disclosure provides highly advantageous anionic citrate-based compositions for controlled binding and release of proteins. In certain embodiments, the present disclosure provides a composition that includes (i) a citrate component, (ii) a polyol, and (iii) an anionic moiety.

Citric acid is an inexpensive, nontoxic, and naturally occurring metabolic molecule that participates in bone anatomy and physiology by regulating the growth of apatite nanocrystals and cellular energy production. Furthermore, citrate-based biomaterials present valuable chemical functional groups for bulk chemistry modification and surface conjugation chemistry. In addition to the citrate chemistry, citrate-based biomaterials provide unique elastomeric properties that can be conducive for native tissue development during regeneration. As used herein, the terms “citrate-based compositions” and “citric-based biomaterials” are generally interchangeable and refer to a polymeric compound produced by reacting citric acid with a diol monomer to generate a polymer having a backbone that comprises hydrolysable ester bond.

Citrate-based biomaterials are rich in carboxyl and hydroxyl bulk chemistry, which can be modified with anionic moiety through monomer addition or surface modification. In exemplary embodiments, the anionic moiety may include one or more of sulfate, sulfonate, phosphate, phosphonate, sulfamate, or carboxylic acid. For example, sulfate and sulfonate containing monomers, including but not limited to taurine or its salt form and isethionic acid or its salt form, can react with the free carboxylic acid groups of citric acid through esterification or amidation reaction.

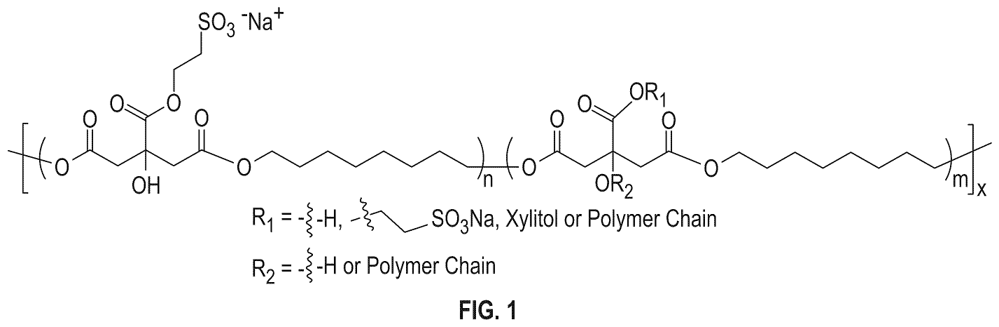

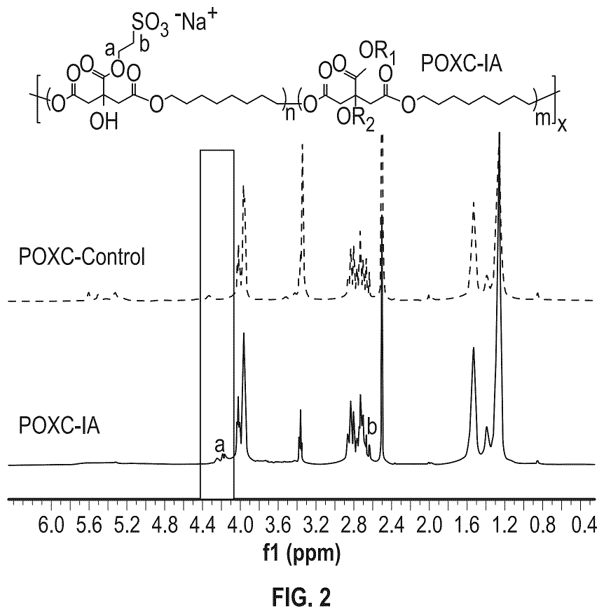

As an example, the addition of isethionic acid sodium salt (IA) to the mixture of citric acid, xylitol, and 1,8-octandiol leads to poly (octamethylene-xylitol citrate) (POXC) with isethionic acid sodium salt to form POXC-IA, the structure of which is shown in FIG. 1 . . . . Proton nuclear magnetic resonance (1H NMR) characterization of the pre-polymer shows successful introduction of the sulfonate moiety in citrate-based polymer, e.g., POXC-IA (see FIG. 2). As shown in FIG. 2, the characteristic peaks from isethionic acid moiety are at 4.22-4.16 ppm and 2.70-2.62 ppm.

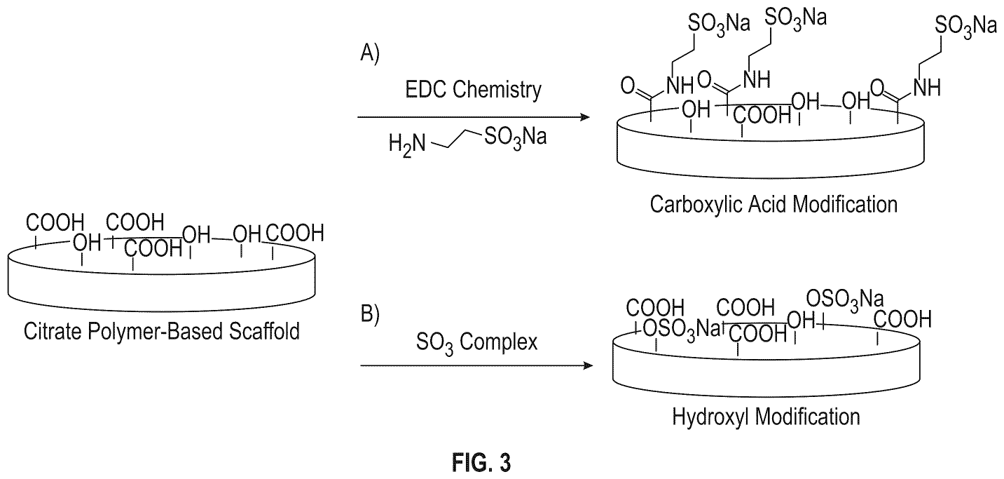

In other exemplary embodiments, sulfates and/or sulfonates can be conjugated to the surface of citrate-based biomaterials using free carboxylic acid (see FIG. 3; conjugation “A”) and/or hydroxyl group (see FIG. 3; conjugation “B”).

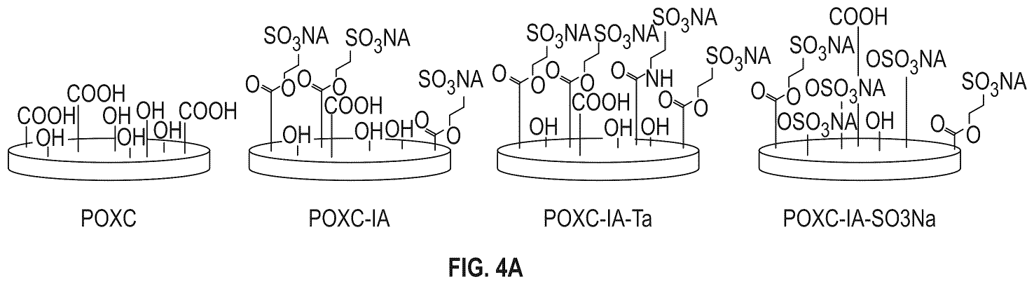

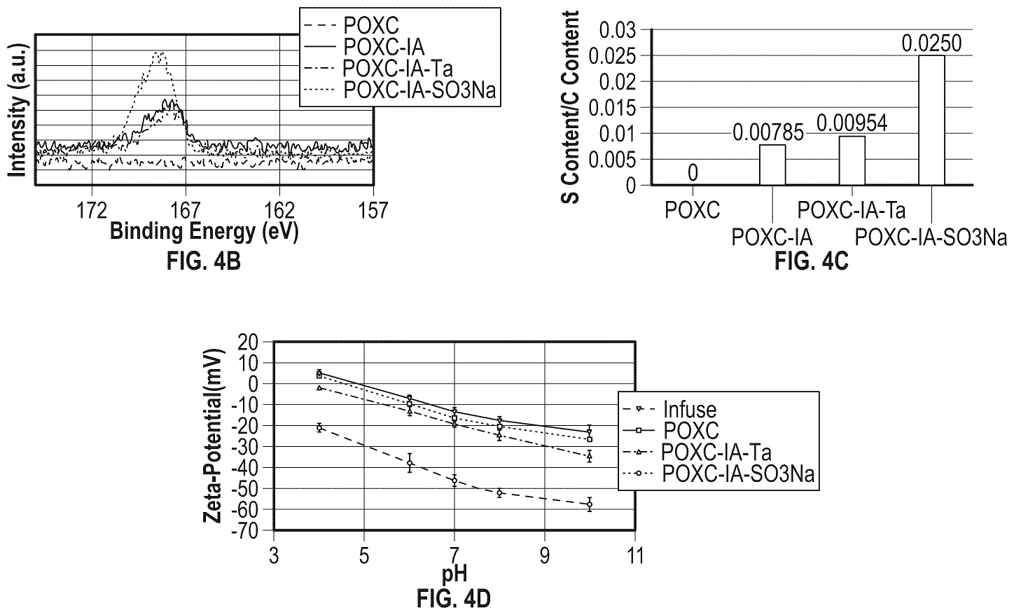

FIG. 4A-FIG. 4D comprise four panels and show exemplary embodiments of modified citrate-based polymer scaffolds with a sulfate and/or sulfonate containing moiety. Specifically, FIG. 4A shows poly (octamethylene-xylitol citrate) (POXC) scaffolds surface modified to present sulfate and/or sulfonate groups. FIG. 4B shows high-resolution X-ray photoelectron spectroscopy (XPS) spectra of S2p. FIG. 4C shows sulfate/sulfonate content obtained from the integration of high-resolution XPS spectra of S2p and C1s, which is defined as the ratio of S atom and C atom contents. The C atom content for POXC-IA-Ta was corrected via the subtraction of the C atom from taurine. FIG. 4D shows surface zeta-potentials of INFUSE collagen, POXC, POXC-IA-Ta and POXC-IA-SO3Na scaffolds under different pHs.

To make porous polymer scaffolds, POXC or POXC-IA pre-polymer was mixed with sodium chloride and then cured at 80° C. for seven (7) days. The salt leached out with deionized water. The obtained POXC-IA scaffolds may be soaked into an 1-ethyl-3-(3-dimethylaminopropyl) carbodiimide (EDC)/N-Hydroxysuccinimide (NHS)/Taurine solution and shaken at room temperature for twenty four (24) hours to form scaffolds with both isethionic acid (IA) and taurine sodium salts (Ta), as shown in FIG. 4A as POXC-IA-Ta. Successful surface conjugation was confirmed by X-ray photoelectron spectroscopy (XPS), which showed an increased sulfur peak intensity (see FIGS. 4B and 4C). As shown in FIG. 4B, the S2p peak centered at about 168 eV corresponds to C—S—O bonds. The sulfonate content can be defined as the ratio of S atom and C atom content (S/C) obtained from the integration of high-resolution XPS spectra of S2p and C1s. As shown in FIG. 4C, the S/C ratio increases from 0 in POXC scaffold to 0.00785 in POXC-IA and to 0.00954 in POXC-IA-Ta.

As another example, surface modification of POXC-IA scaffolds can be achieved by soaking in a sulfur trioxide triethylamine complex in DMF at 35° C. for twenty four (24) hours. The substitution reaction on the pendant hydroxyl groups leads to scaffolds with both isethionic acid and sulfate groups, as shown in FIG. 4D, POXC-IA-SO3Na. As shown in FIGS. 4B and 4C, XPS characterization confirms successful surface modification with a significant increase in the sulfate and sulfonate content. As shown in FIG. 4B, the POXC-IA-SO3Na shows a strong S2p peak centered at about 168 eV. As shown in FIG. 4C, the S/C ratio in POXC-IA-SO3Na increases to 0.250.

In exemplary embodiments, the sulfate/sulfonate contents may be tuned by the amount of sulfation reagents and/or by reaction temperature. The content of sulfate/sulfonate effectively modulates the surface charges of those citrate-polymers-based scaffolds. As shown in FIG. 4D, the surface zeta-potential increases with the sulfate/sulfonate content under pH 4 to 10. It is noteworthy that the surface zeta-potentials of POXC-based scaffolds are higher than the commercially available INFUSE collagen sponge (Medtronic plc, Minneapolis, MN).

To show the ability of sulfate and/or sulfonate modified citrate-based materials to bind and release proteins, recombinant human BMP-2 (rhBMP-2) is used as a model protein in an experimental test. Specifically, 1 μg of rhBMP-2 was loaded onto INFUSE collagen scaffolds, POXC scaffold, and sulfation/sulfonation POXC scaffolds modified with different sulfation/sulfonation degree. Cumulative rhBMP-2 release is then measured at 1, 2, 3, 7, 14, and twenty one (21) days using a BMP-2 ELISA Kit (R&D Systems, Minneapolis, MN).

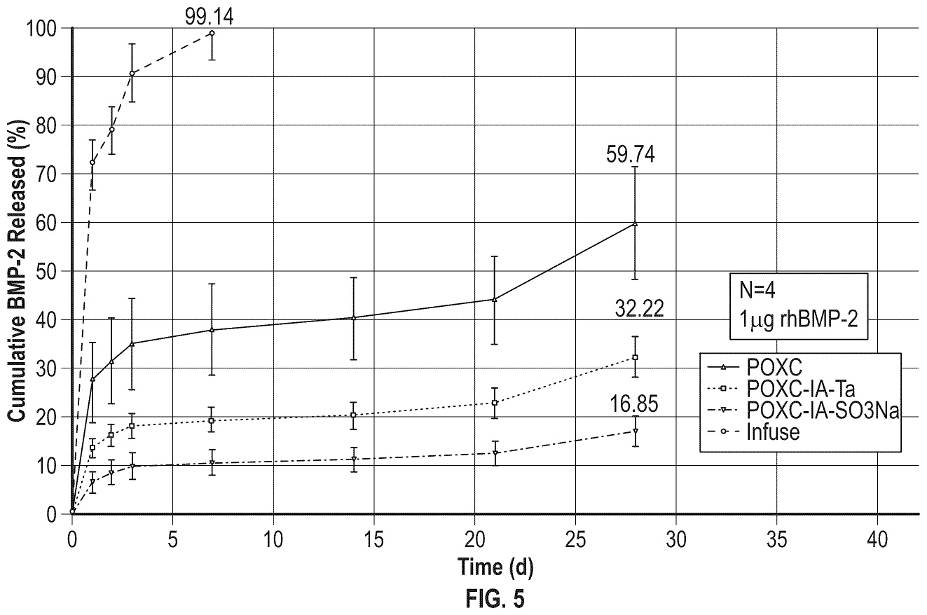

As shown in FIG. 5, rhBMP-2 release from INFUSE collagen scaffolds resulted in an immediate burst release of 71.17±5.16% after twenty four (24) hours. After seven (7) days, 99.14±5.72% is released from the INFUSE collagen scaffolds. In contrast, rhBMP-2 release from POXC scaffold and modified POXC scaffolds is slower and controlled with the degree of sulfation/sulfonation. The measured results are consistent with the surface zeta-potentials, confirming the surface charge plays a key role in controlling of rhBMP-2 binding and releasing. For example, as shown in FIG. 5, after twenty four (24) hours, rhBMP-2 release is 27.02±8.26%, 13.42±2.01%, and 6.33±2.29% for POXC, POXC-IA-Ta (S/C=0.00954) and POXC-IA-SO3Na (S/C=0.0250) scaffolds, respectively. A slow and steady rhBMP-2 release continued after twenty eight (28) days, with only 59.74±11.71% of rhBMP-2 released from the POXC, of 32.22±4.26% of rhBMP-2 released from the POXC-IA-Ta scaffolds, and 16.85±2.97% released from the POXC-IA-SO3Na scaffolds, respectively.

As shown in FIG. 5, the unmodified POXC scaffolds are also able to sequester the BMP-2 more effectively than the absorbable collagen sponge used in INFUSE.

In some exemplary embodiments, the disclosed composition for binding protein for regenerative engineering includes (i) a citrate component, and (ii) a polyol. The experimental data presented herein demonstrates the tunability of release by the addition of other negatively charged groups such as sulfates/sulfonates.

Based on the experimental results presented herein, one of ordinary skill can expect similar outcomes using monomers containing other anions including, but not limited to, phosphates, phosphonates, sulfamates, and carboxylates. This is due to the ability of these anions to increase the surface zeta potential in a similar fashion to the sulfates and sulfonates presented in this disclosure. The BMP-2 release data shows a direct correlation to the zeta potential on the scaffold surface. Finally, a skilled practitioner can also expect other heparin-binding growth factors, such as bFGF, TGF-β1, VEGF, PDGF, and EGF, to be sequestered by the chemistries presented above.

Although the present disclosure has been described with reference to exemplary embodiments and implementations, the present disclosure is not limited by or to such exemplary embodiments/implementations.

1. A composition comprising:
(i) a citrate component,
(ii) a polyol, and
(iii) an anionic moiety.

2. The composition of claim 1, wherein the citrate component comprises one or more of citric acid, citrate, or ester of citric acid.

3. The composition of claim 1, wherein the polyol comprises a diol.

4. The composition of claim 3, wherein the diol comprises one or more of butanediol, hexanediol, octanediol, or polyethylene glycerol.

5. The composition of claim 1, wherein the polyol comprises one or more of glycerol, beta-glycerol phosphate, or xylitol.

6. The composition of claim 1, wherein the anionic moiety comprises one or more of sulfate, sulfonate, phosphate, phosphonate, sulfamate, or carboxylic acid.

7. The composition of claim 6, wherein the sulfate and/or sulfonate containing moiety further comprises one or more of hydroxyl, amine, or carboxylic acid.

8. The composition of claim 6, wherein the sulfate and/or sulfonate containing moiety comprises one or more of taurine, isethionic acid, or sodium 2-carboxyethane-1-sulfonate.

9. The composition of claim 6, wherein the sulfate and/or sulfonate containing moiety is conjugated via amidation reaction, esterification, and/or substitution reaction with a sulfur trioxide complex.

10. The composition of claim 9, wherein the amidation reaction is assisted by 1-ethyl-3-(-3-dimethylaminopropyl) carbodiimide hydrocholoride (EDC).

11. The composition of claim 6, wherein the phosphate and phosphonate containing moiety comprises one or more of hydroxyl, amine, or carboxylic acid.

12. The composition of claim 11, wherein the phosphate and phosphonate containing moiety comprises 3-(phosphonooxy) propanoic acid or 2-hydroxyethyl phosphate.

13. The composition of claim 6, wherein the anionic moiety further comprises one or more of alkene, alkyne, halogen, borate, or azide.

14. The composition of claim 1, wherein a peptide is conjugated to a surface of the composition.

15. The composition of claim 14, wherein the peptide is a protein binding peptide.

16. The composition of claim 14, wherein the peptide is a protein mimicking peptide.

17. The composition of claim 1, wherein the composition further comprises a particulate inorganic material.

18. The composition of claim 17, wherein the particulate inorganic material comprises one or more of hydroxyapatite, tricalcium phosphate, biphasic calcium phosphate, or Bioglass.

19. The composition of claim 17, wherein the particulate inorganic material is present in an amount between 10 and 60 wt.-% of the composition.

20. The composition of claim 17, wherein the particulate inorganic material is micro or nano-sized.

21. The composition of claim 17, wherein the particulate inorganic material is rod-shaped.

22. The composition of claim 17, wherein the particulate inorganic material is fiber-shaped.

23. The composition of claim 1, wherein the citrate component and polyol react to form a polymer.

24. A scaffold formed at least in part from the composition of claim 1, wherein the scaffold is biodegradable.

25. The composition of claim 22, wherein the scaffold is 50-90% porous.

26. The composition of claim 22, wherein the scaffold contains a gradient porous structure.

27. The composition of claim 1, wherein the composition binds proteins through electrostatic interactions.

28. The composition of claim 27, wherein the protein is one or more of bone morphogenic protein (BMP), basic fibroblast growth factor (bFGF), transforming growth factor beta (TGF-β1), vascular endothelial growth factor (VEGF), platelet derived growth factor (PDGF), and/or epidermal growth factor (EGF).

29. The composition of claim 28, wherein the BMP comprises one or more of BMP-2, BMP-4, or BMP-7.
